(19) United States
(12) Patent Application Publication
(10) Pub. No.: US 2016/0008514 A1
(43) Pub. Date: Jan. 14, 2016

(54) Soft Tissue Pouch and Methods of Use Thereof

(71) Applicant: LIFENET HEALTH, Virginia Beach,, VA (US)

(72) Inventor: Alyce Linthurst JONES, Virginia Beach, VA (US)

(73) Assignee: LifeNet Health, Virginia Beach, VA (US)

(21) Appl. No.: 14/776,153

(22) PCT Filed: Mar. 17, 2014
(86) PCT No.: PCT/US2014/030232

§ 371 Date: Sep. 14, 2015

Related U.S. Application Data
(60) Provisional application No. 61/787,368, filed on Mar. 15, 2013.

(51) Int. Cl. A61L 27/36; A61L 27/54; A61L 29/00; A61L 29/16; A61L 31/00; A61L 31/16

(52) U.S. Cl. CPC A61L 27/3683; A61L 31/005; A61L 31/16; A61L 29/005; A61L 29/16; A61L 27/362; A61L 27/54; A61L 27/3625; A61L 2300/404; A61L 2400/18

(57) ABSTRACT

The invention relates to a soft tissue pouch and methods of use thereof.

### BACKGROUND OF THE INVENTION

The invention relates to a soft tissue pouch and methods of use thereof.

### SUMMARY OF THE INVENTION

The invention relates to a soft tissue pouch that is configured to encapsulate a medical device. The invention also relates to a medical product comprising a medical device and a soft tissue pouch encapsulating the same.

The invention also relates to methods of preparing the soft tissue pouch product. In some embodiments, the method of preparing the soft tissue pouch product comprises obtaining a mammalian soft tissue, preferably a sheet format; decellularizing and/or devitalizing the tissue; optionally treating the tissue with a plasticizer; suturing, stapling or gluing the soft tissue into a pouch shape configured to encapsulate a medical device; packaging the soft tissue pouch in a packaging material; and optionally sterilizing the product.

The invention relates to a method of implanting a medical device into a patient. In some embodiments, the method of implanting the medical device into a patient comprises (i) encapsulating the medical device with a soft tissue sheet or inserting the medical device into a soft tissue pouch; (ii) closing the opening of soft tissue pouch; and (iii) implanting the medical device encapsulated with the soft tissue pouch.

### BRIEF DESCRIPTION OF THE DRAWINGS

FIGS. 1A, 1B, and 2C depict different views of an exemplary soft tissue pouch.

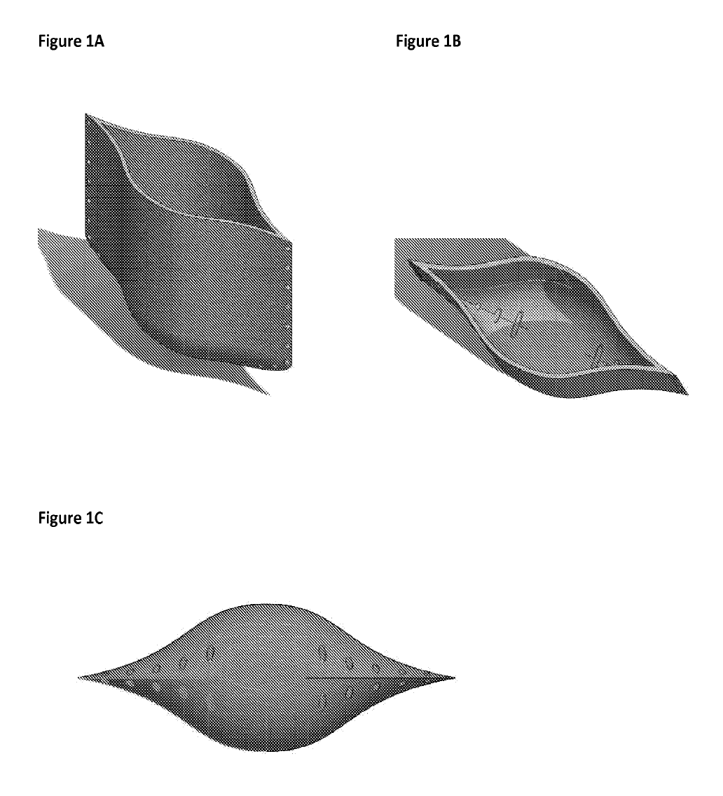

FIGS. 2A and 2B depict an exemplary medical product comprising a medical device and a soft tissue pouch encapsulating the same prior to suturing.

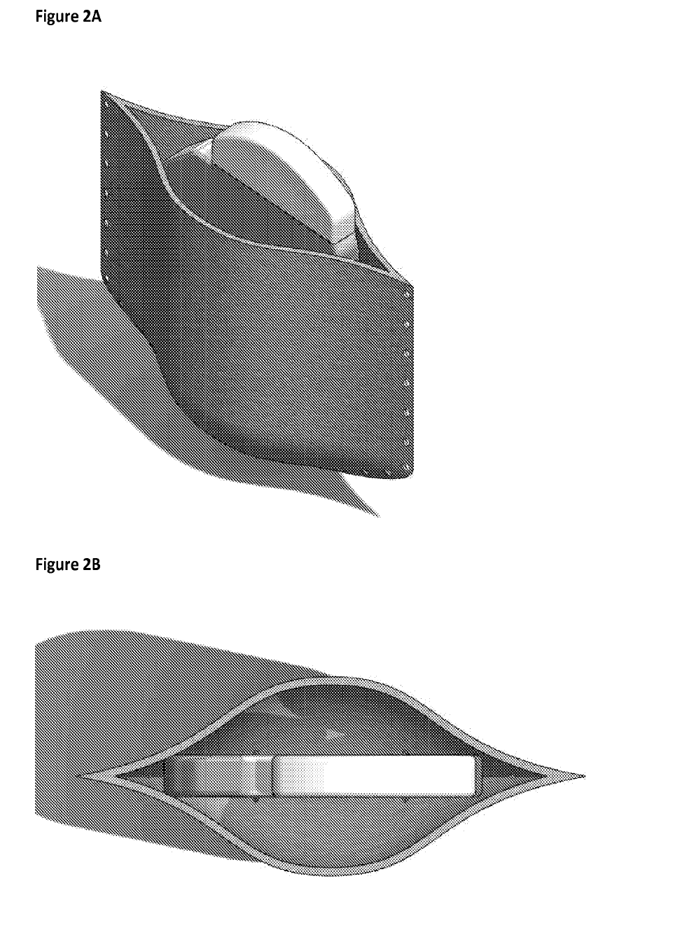

FIG. 3 depicts another exemplary soft tissue pouch ready to encapsulate medical devices of various shapes with possible suture sites.

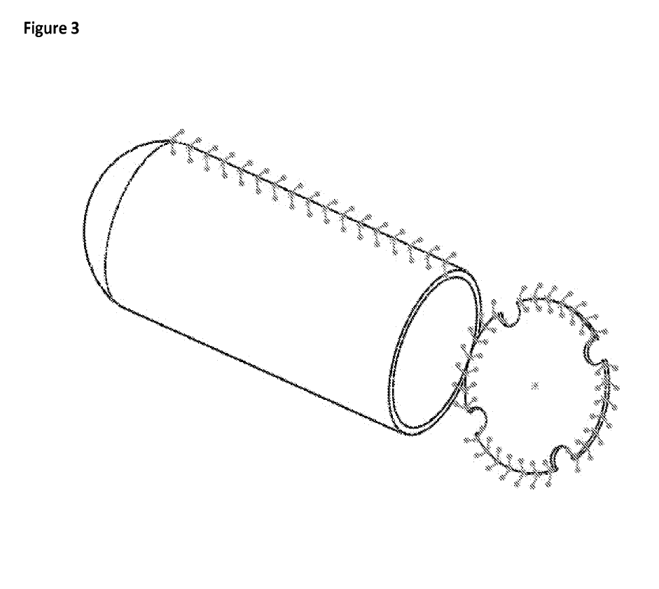

### DETAILED DESCRIPTION OF THE INVENTION

The invention relates to a medical product comprising a medical device and a soft tissue pouch encapsulating the same.

A medical product refers to any biological product, derived from human or animal that can be implanted in a subject. A medical device refers to any instrument, apparatus, implement, machine, appliance, or implant for in vivo use, material or other similar or related article, intended by the manufacturer to be used, alone or in combination, in or on a subject. In some embodiments, the medical device may have specific medical purpose(s) of: diagnosis, prevention, monitoring, treatment or alleviation of disease, diagnosis, monitoring, treatment, alleviation of or compensation for an injury, investigation, replacement, modification, or support of the anatomy or of a physiological process, supporting or sustaining life, control of conception, disinfection of medical devices, or providing information by means of in vitro examination of specimens derived from the human body; and does not achieve its primary intended action by pharmacological, immunological or metabolic means, in or on the human body, but which may be assisted in its intended function by such means. For example, the medical device may be a pacemaker, an ICD, an insulin pump, breast implant or an indwelling catheter.

A soft tissue pouch refers to any soft tissue materials suitable for covering the surface of the medical device. In some embodiments, the soft tissue pouch may cover the surface of the medical device by about 100%, 99%, 98%, 97%, 96%, 95%, 90%, 85%, 80%, 75%, 70%, 65%, 60%, 55%, 50% or more. In some embodiments, the soft tissue is selected from skin, dermis, pericardium, fascia, arteries or veins, dura mata, ammonic membrane, bladder, small or large intestine. In additional embodiments, the soft tissue described herein is an autograft, an allograft, or a xenograft. In further embodiments, the soft tissue pouch has a thickness of about 0.005 mm, 0.05 mm, 0.1 mm, 0.2 mm, 0.3 mm, 0.4 mm, 0.5 mm, 1 mm or more. In yet further embodiments, the soft tissue pouch has a thickness of about 0.1 mm, 0.2 mm, 0.3 mm, 0.4 mm, 0.5 mm, 1.5 mm, 2.0 mm, 2.5 mm or less.

As used herein, the term “about” modifying, for example, the quantity of an ingredient in a composition, concentrations, volumes, process temperature, process time, yields, flow rates, pressures, and like values, and ranges thereof, refers to variation in the numerical quantity that can occur, for example, through typical measuring and handling procedures used for making compounds, compositions, concentrates or use formulations; through inadvertent error in these procedures; through differences in the manufacture, source, or purity of starting materials or ingredients used to carry out the methods; and like considerations. The term “about” also encompasses amounts that differ due to aging of, for example, a composition, formulation, or cell culture with a particular initial concentration or mixture, and amounts that differ due to mixing or processing a composition or formulation with a particular initial concentration or mixture. Whether modified by the term “about” the claims appended hereto include equivalents to these quantities. The term “about” further may refer to a range of values that are similar to the stated reference value. In certain embodiments, the term “about” refers to a range of values that fall within 10, 9, 8,7, 6, 5,4, 3, 2, 1 percent or less of the stated reference value.

In one aspect, the soft tissue pouch prevents infection of the encapsulated medical device. In another aspect, such prevention lasts for the entire implant life of the said medical device. In some embodiments, in the soft tissue pouch, the epidermal basement membrane side of the soft tissue is placed outward (e.g. to face host tissue) and the reticular surface is facing the medical implant. In other words, the outer surface of the soft tissue pouch may comprise the epidermal basement membrane of the soft tissue. As the basement is a biological barrier to microbes and other adventitious agents, having the epidermal basement membrane facing the body wall may act as a barrier to microorganisms. This may be advantageous as indwelling medical devices are susceptible to bacterial colonization over their indwelling life span, for example, as a result of contamination at time of implant, or subsequent procedures the patient may undergo, such as dental cleaning and/or extractions that may result in a bolus of bacteria entering the bloodstream. Moreover, having the epidermal basement membrane facing the body wall may allow the medical product described herein to integrate into the body wall and serve to stabilize the device to prevent device migration. In another aspect, the soft tissue pouch may prevent migration of the encapsulated medical device in a host.

The invention also relates to methods of preparing the soft tissue pouch product. In some embodiments, the method of preparing the soft tissue pouch product comprises obtaining a soft tissue; optionally decellularizing and/or devitalizing the soft tissue; optionally treating the soft tissue with a plasticizer; suturing, stapling or gluing the soft tissue into a pouch shape configured to encapsulate a medical device; packaging the soft tissue pouch in a packaging material; and optionally sterilizing the product. The invention also relates to medical devices produced by the methods described herein.

In one aspect, the soft tissue may be a mammalian soft tissue in the form of a sheet. In another aspect, the soft tissue in the soft tissue pouch may be decellularized and/or devitalized. For example, the soft tissue may be decellularized and/or devitalized by methods described in U.S. Pat. Nos. 6,743,574 and 7,338,757, which is incorporated by reference in its entirety herein. In some aspects, the decellularization and/or devitalization of the soft tissue may allow the soft tissue pouch to integrate into the implant site or into the recipient because the cells from the implant site or the recipient will grow into the pouch and remodel the pouch over time. In another aspect, the soft tissue may be treated with a plasticizer by methods described in U.S. Pat. No. 6,569,200, which is incorporated by reference in its entirety herein.

In some embodiments, after decellularization and/or devitalization treatment, the tissue may be fashioned and sutured into an appropriately sized pouch for the device, packaged, sterilized and/or stored until shipment. At the time of implant, the device will be diluted in a solution by methods described in U.S. Pat. No. 6,569,200, and then the surgeon will place the device into the pouch, suture it closed and implant the device.

In some embodiments, after decellularization and/or devitalization treatment, the tissue may be fashioned and sutured into an appropriately sized pouch for the device, packaged, sterilized and/or stored until shipment. At the time of implant, the surgeon will place the device into the pouch, suture it closed and implant the device.

Methods of the current invention may include sterilization of soft tissue, soft tissue graft, medical device, and/or sterilization of packaged medical product. Sterilization may be performed using methods known in the art. The sterilization may involve the use of ionizing radiation, in some embodiments. In certain embodiments, the absorbed dose of ionizing radiation is between about 8.0 KGy and about 50 KGy, between about 8.0 KGy and about 25 KGy, and between about 8.0 KGy and about 20 KGy in some embodiments, the sterilizing step includes placing the packaged composition on dry ice and irradiating the packaged composition. In certain embodiments, sterilization is performed at a temperature of between about −20° C. and −70° C.

Methods of the current invention may include sterilization of soft tissue, soft tissue graft, medical device, and/or sterilization of packaged medical product. Sterilization may be performed using methods known in the art. The sterilization may involve the use of chlorine dioxide. The pouch is placed into pouch with a breathable header and placed into the sterilization chamber. The zeolith containing the chlorine dioxide is activated by addition water and provides a sterilizing dose of chlorine dioxide. In certain embodiments, the sterilizing dose is between about 0.001% to about 1.0% v/v chlorine dioxide, and between about 0.01% to about 0.1% chlorine dioxide. Once the contact time as elapsed, the breathable header is removed.

The invention relates to a method of implanting a medical device to a patient. In some embodiments, the method of implanting the medical device to a patient comprises (i) encapsulating the medical device with a soft tissue sheet or inserting the medical device into a soft tissue pouch; (ii) closing the opening of soft tissue pouch; and (iii) implanting the medical device encapsulated with the soft tissue pouch.

### EXAMPLE 1

Two heart valves were decellularized in accordance to the disclosure in U.S. Pat. No. 6,743,574. One was turned inside out placing the basement membrane to the bulk solution, and the other in its native conformation placing the adventitia to the bulk solution. The one with the basement membrane outside did not decellularize as assessed by DNA and H&E staining, unlike the one in the native conformation which passed the acceptance criteria. The heart valve with the basement membrane facing the bulk solution had a residual DNA content of 250 ng DNA/mg wet weight of tissue as assessed by pico green whereas the heart valve with the adventitial surface facing the bulk solution had a residual DNA content of 2 ng DNA/mg wet weight of tissue as assessed by pico green. The heart valve with the basement membrane facing the bulk solution had residual eosinophilic staining material known to be nuclear in origin whereas the heart valve with the adventitial surface facing the bulk solution had no discernible eosinophilic staining. It was shown that the basement membrane served as a barrier and did not allow the detergent solution and endonuclease to penetrate the basement membrane to facilitate decellularization. This was an unexpected result as detergents lower the surface tension of solutions thereby increasing their ability to penetrate and to solubilize proteins and lipids that constitute cell membranes. It was anticipated that the detergent would be able to penetrate the basement membrane and facilitate the penetration endonuclease to degrade the DNA.

### EXAMPLE 2

One partial thickness defect was created (approximately 1 cm×2 cm) in vivo in the abdominal wall of a rat without piercing into the abdominal cavity so that the peritoneum remained intact. A 1 cm×2 cm piece of either Parietex or DermACELL® mesh (devitalized human dermis) was placed over the defect and sutured to the muscle layer. The DermACELL was implanted so that the dermal side (reticular) was adjacent to the abdominal muscle wall, so that the basement membrane would receive the innoculm. Each animal then received 0.050 mL of Staphylococcus aureus (S. aureus) ATCC #25923 at one of four different dosages between 1×10⁵ and 1×10⁸ CFU/implant site directly applied to the exposed ventral aspect of the implanted Parietex or DermACELL® mesh. For each of the four doses of bacteria, there were 3 animals in the DermACELL group and 3 animals in the Parietex group, for a total of 24 animals. After 28 days, the implanted mesh materials were removed, and one half of each sample was processed to determine the number and type of bacteria remaining in the mesh. The samples were put through repeated vortex/sonication in Tween 80, diluted, plated on trypticase soy agar, incubated at 37±2° C. for up to 72 hours, enumerated and indentified. The other half of each sample was processed for H&E histology.

The results showed that a dose of 1×10⁵ CFU of S. aureus strain ATCC #25923/implant site was effective in producing an infection in the majority of Parietex implant sites without causing morbidity or mortality. At a dose of 1×10⁵ CFU/implant site, all three implant sites repaired with Parietex retained high counts of S. aureus, while all three implant sites repaired with DermACELL® displayed no measureable counts of S. aureu, indicating a 100% clearance rate. Thus, at this dose, the number of S. aureus recovered from Parietex™ (negative control implant) was sufficiently large to discern a substantial difference in bacterial recovery when compared to DermACELL® or other test articles that similarly resist infection.

EXAMPLE 3

Three pieces of cardiovascular tissue, pulmonary artery, were seeded on the basement membrane surface and three were seeded on the adventitial (body wall facing) surface with 10-100 CFU of Bacillus atropheous. The tissue was then extracted, the extract was plated on TSA and incubated at 37° C. +/−2° C. for 5-7 days. The tissues seeded on the basement membrane surface resulted in no organisms being recovered, and the tissues seeded on the adventitial surface had 87% of the organisms recovered. These results support that the basement membrane more specifically, those that were previously lined by endothelial cells and/or epithelial cells in soft tissues are antimicrobial.

1. A medical product comprising a medical device and a soft tissue pouch encapsulating the medical device.

2. The medical product according to claim 1, wherein the soft tissue is selected from skin, dermis, pericardium, fascia, arteries or veins, dura mata, ammonic membrane, bladder, small or large intestine.

3. The medical product according to any of claims 1-2, wherein the soft tissue is an autograft, an allograft, or a xenograft.

4. The medical product according to any of claims 1-3, wherein the soft tissue is dermis.

5. The medical product according to any of claims 1-4, wherein the basement membrane side of the soft tissue is placed outward.

6. The medical product according to any of claims 1-5, wherein the medical device is a pacemaker, an ICD, an insulin pump, or an indwelling catheter.

7. A method for preparing a soft tissue pouch for implanting a medical device into a patient, comprising:
(i) obtaining a mammalian soft tissue;
(ii) optionally decellularizing and/or devitalizing the soft tissue;
(iii) optionally treating the soft tissue with a plasticizer;
(iv) suturing, stapling or gluing the soft tissue into a pouch shape configured to encapsulate a medical device;
(v) packaging the soft tissue pouch in a packaging material; and
(vi) optionally sterilizing the product.

8. A method for implanting a medical device into a patient, comprising
(i) encapsulating the medical device into a soft tissue pouch;
(ii) closing the opening of soft tissue pouch; and
(iii) implanting the medical device with the soft tissue pouch.

9. The method according to claim 7 or 8, wherein the soft tissue is selected from skin, dermis, pericardium, fascia, arteries or veins, dura mata, ammonic membrane, bladder, small or large intestine.

10. The method according to any of claims 7-9, wherein the soft tissue is an autograft, an allograft, or a xenograft.

11. The method according to any of claims 7-10, wherein the soft tissue is dermis.

12. The method according to any of claims 7-11, wherein the basement membrane side of the soft tissue is place outward to face the host tissue.

13. The method according to any of claims 7-12, wherein the medical device is a pacemaker, an ICD, an insulin pump, or an indwelling catheter.

14. The method according to any of claims 7-13, wherein the soft tissue pouch is prevents infection of the medical device encapsulated therein.

15. The method according to any of claims 7-13, wherein the soft tissue pouch prevents migration of the medical device encapsulated therein in a host.

16. A medical product produced by the method according to claim 7.
